(19) United States
(12) Patent Application Publication
(10) Pub. No.: US 2004/0255936 A1
(43) Pub. Date: Dec. 23, 2004

(54) Disposable metered dose inhaler with integrated electronic counter

(76) Inventor: David Urbanus, North Easton, MA (US)

Correspondence Address: CHARLES G. CALL, 68 HORSE POND ROAD, WEST YARMOUTH, MA, 02673-2516, US

(21) Appl. No.: 10/836,174

(22) Filed: Apr. 30, 2004

Related U.S. Application Data
(60) Provisional application No. 60/466,906, filed on Apr. 30, 2003.

(51) Int. Cl. A61M 11/00; A61M 15/00; A61M 16/00

(52) U.S. Cl. 128/200.23; 128/203.15

(57) ABSTRACT

A disposable medication inhaler consisting of a medication canister permanently attached to a disposable housing which also encloses electronic counting and display circuitry for indicating the number of doses of medication remaining in the counter, as well as the number of doses administered in a given time period such as the current day or the previous 24 hours. Input means are included for presetting the electronic dose counter to equal the number of doses in the canister at the time the dispenser is assembled. The housing and electronics are disposed with the attached canister when empty.

### CROSS REFERENCE TO RELATED APPLICATIONS

This application is a Non-Provisional of, claims the benefit of the filing date of, and incorporates by reference the disclosure of: U.S. Patent Application Ser. No. 60/466,906 filed on Apr. 30, 2003.

This application, and the foregoing provisional application, describe an improvement over the Dispenser Monitor System described in U.S. Pat. No. 5,505,192 issued to Samiotes et al. on Apr. 9, 1996, the disclosure of which is also incorporated herein by reference.

### FIELD OF THE INVENTION

This invention relates to an integrated medication monitoring counter for a medication dispenser, such as an inhalation device for asthma medication of a dispensing device for medication to treat diabetes.

### BACKGROUND OF THE INVENTION

Asthma is one of the fastest growing ailments in the United States. Over 50 million people require some form of treatment for asthma or related allergies; about one in five Americans Asthma and allergies are the most common chronic diseases in this country. Bronchitis and emphysema are also part of this category of ailments.

The consequences of an untreated asthma attack can be deadly. A serious asthma attack can result in a combination of swelling of the lungs and muscular constriction of the airways with little or no air movement beyond the obstruction. Each year in the United States an estimated seven thousand deaths occur due to asthma alone. A key factor putting the patient at risk is the failure or the inability to comply with the medication program suggested by the doctor or just as a result of an empty medication canister.

There are two basic types of medications that are used in the treatment of asthma: bronchodialator, a quick acting asthmatic medication, or inhaled steroids, a slower acting drug. These bronchodialator medications are fast acting medications that sometimes are referred to as “rescue medications”.

The most common and effective administration of respiratory medications is through the use of a Metered Dose Inhaler (MDI). The MDI device currently in use is comprised of an L-shaped dispenser (usually plastic) and an aerosol canister (metal). The aerosol canister contains medication and is capable of delivering a finite number of metered doses of medication. The aerosol canister is inserted into the L-shaped dispenser. The canister is installed in an applicator, which receives the canister in inverted orientation. The bottom of the canister is facing upward. The patient self-administers the medication. A single push on the canister releases a single dose of the medication in the form of an inhaled aerosol

A significant deficiency of MDI's is the inability of the patient to precisely determine how many metered doses of medication remain in the aerosol canister. The metered dose inhaler canister is always a metal container, and so it is not possible to tell accurately how much medicine or metered dosages are remaining. The patient, unless he or she records the information in a diary, has no real chance to remember what has been taken on any given day or how many dose of medication are remaining in the medication canister. The patient is at risk to run out at an inopportune time, and is in medical jeopardy including shortness of breath, choking, respiratory discomfort, and even hospitalization.

In addition to administration of respiratory medication, MDI's are being tested for the administration of medications to treat diabetes. The inherent limitations of the inability to identify the number of doses remaining in the medication canister portion of the MDI are exacerbated by the possible severe and well-documented consequences of a diabetic being unaware of the correct amount of needed medication at critical times.

There is currently no simple procedure to determine the number of metered doses of medication remaining in the aerosol canister. There are various recommendations given by doctors and pharmacists: one method directs the patient to float the medication canister; the other method is to shake the canister to see if there is medication in the canister. These methods are inexact methods at best. The amount of propellant remaining in the canister will be disproportionate to the actual medication that is in the canister; (This fact is documented by all the manufacturers of the medication canisters), this could give the patient an incorrect assurance that there is still medication remaining in the canister when in fact only inert propellant is remaining in the canister.

There have been some attempts to at add a mechanical counting mechanism to MDIs but these mechanisms have proven to be inaccurate and unreliable, thus perpetuating the current situation for monitoring the doses remaining in the medication canister.

U.S. Pat. No. 5,505,192 issued to Samiotes et al. on Apr. 9, 1996, the disclosure of which is incorporated herein by reference, describes an MDI that includes a dispenser monitoring circuit including a switching device responsive to actuation of the dispenser container; a counter circuit, responsive to the switching device, for registering that a quantity of material has been dispensed; and a display device responsive to the counter circuit for indicating the status of the material in the dispenser. The dispenser described in the Samiotes et al. patent accepts a cylindrical canister that holds the medication to be dispensed. When the contents of the canister are depleted, the canister can be replaced, and pushbuttons on the exterior of the dispenser may be manipulated by the user to reset the counter circuit to a number equivalent to the dose capacity of the new canister.

The MDI dispenser described in the Samiotes et al. patent has proven to be highly effective, but it suffers from several disadvantages. When it becomes necessary to replace the canister, the user must obtain a replacement size of compatible size and configuration. The need to purchase a matching replacement cartridge may be difficult or confusing for many consumers. In addition, when the new canister in inserted into the dispenser, the consumer must reset the dispenser's counter to the proper number of doses, a process which may also be difficult or confusing due to the need to press combinations of buttons (e.g. the “set” and “clear” buttons of the Samiotes et al. device), and will typically require the user to retain and again read an instruction manual. Still further, the presence of these buttons on the exterior of the housing raises the risk that the user will inadvertently reset the counter, thus loosing the ability to estimate the current content of the canister present in the dispenser. Finally, the need to provide these additional user-operated switches increases the cost of the device.

The prior MDI dispenser described in the Samiotes et al. patent includes a user-operated “history” switch which the user can depress to display a count of the number of doses dispensed within a particular time interval; for example, the number dispensed during the current day. While this display has proven to be very useful to the consumer in monitoring the extent to which the MDI has been used on a daily basis, the presence of the history switch and the need to understand its use further complicates the use of the device by the consumer.

### SUMMARY OF THE INVENTION

It is an object of the present invention to provide a simplified, low-cost medication dispensing system comprising the integrated combination of a medicine canister which is permanently attached to a canister housing which also houses an electronic counting and dosage monitoring system. In accordance with the invention, the disposable canister housing and the canister and counting electronics which are contained in the housing, are simply discarded when the contents of the canister are depleted.

The preferred embodiment of the invention takes the form of a disposable medication dispenser comprising the combination of a canister for storing medication, an electronic circuit for displaying the number of doses remaining in the canister, and a housing for the electronic circuit permanently attached to the canister, the housing and electronic circuit being discarded with the canister when the medication in said canister have been depleted.

In accordance with the invention, numerical display preferably shows the number of doses administered from the canister during an immediately preceding time interval, such as the current day or the preceding 24 hour period.

Electrical switching devices which may be used to preset the counter when the disposable dispenser is assembled and may are preferably positioned, on the circuit board upon which the other components of the electronic counter circuit are mounted. These count presetting switches are not accessible to the person using the dispenser after it has been assembled, thus eliminating the possibility that the count may be inadvertently changed during normal use, and simplifying the operation of the unit by eliminating the need for the consumer to be concerned with or to operate the count presetting switches. In addition, the cost of more expensive user-operated switches is eliminated.

In accordance with a further feature of the present invention, the “history” switch used in the device described in the above-noted Samiotes patent may be eliminated by employing an LCD output display which concurrently shows both the number of doses remaining in the canister and the number of doses already administered on a given day. In this way, both displays are always presented to the user, and there is no need to learn about or manipulate the additional “history” switch.

In accordance with still another feature of the invention, the LCD display showing the number of doses remaining in the canister and the number of doses previously administered on the current day is placed on the canister housing directly above the mouthpiece so that it faces the user and can be easily seen when the dispenser is brought to the mouth. The only operation the user need perform is pressing the canister downward within the housing to cause the medication to be dispensed. No other user function, other than simply dispensing the medicine, need be learned or performed.

### BRIEF DESCRIPTION OF THE DRAWINGS

In the detailed description which follows, frequent reference will be made to the attached drawings, in which:

FIG. 1 is a perspective view of a Metered Dose Inhaler (MDI) that embodies the invention;

FIG. 2 is an exploded view of MDI seen in FIG. 1;

FIG. 3 is a cross-sectional view of the entire MDI system;

FIG. 4 is a front elevational view of the MDI;

FIG. 5 is a side elevational view of the MDI;

FIG. 6 is an overhead or top plan view of the MDI;

FIG. 7 is a diagram illustrating the method used for setting, recording and displaying the medication canister dose count throughout the monitoring process;

FIG. 8 is a diagram illustrating the method used for setting, recording, and storing data indicating the history of daily doses taken, and displaying the medication canister 7 dose count during the monitoring process; and

FIG. 9 shows several of the key components of the electronic printed circuit board module used to implement the invention.

### DETAILED DESCRIPTION

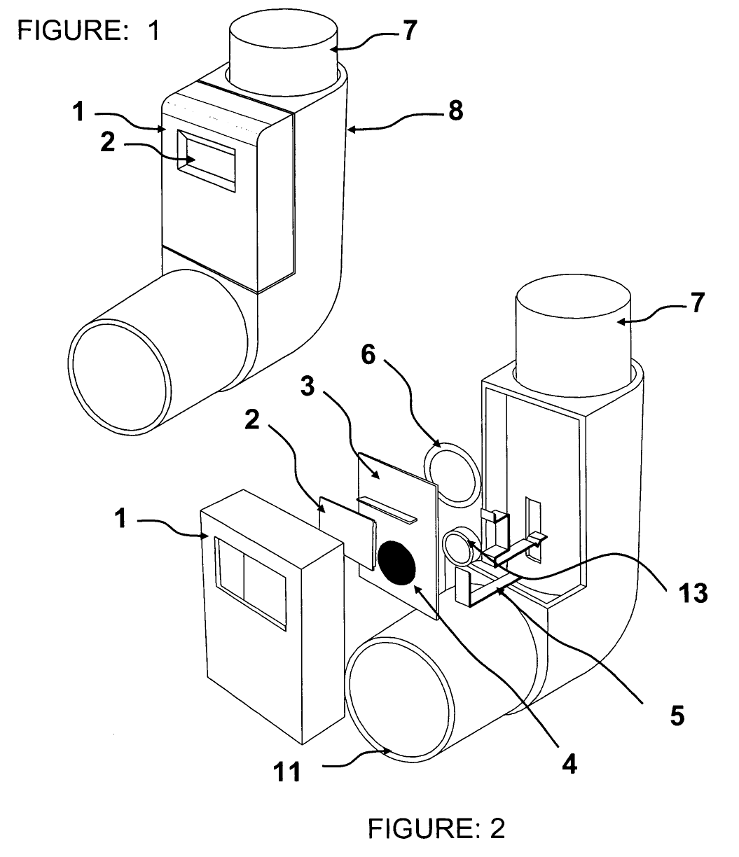

FIG. 1 illustrates a totally disposable (MDI) Metered Dose Inhaler 8 that incorporates ergonomic features that aid the patient's use of the medication system. The shape of the plastic housing 8 is ergonomically designed to fit comfortably in the hands of a 98th percentile of prospective users. The digital display seen at 2 is uniquely designed to be viewed clearly from a full 150 degrees angle. The user dispenses medication from the dispenser by depressing the medication canister 7 at its top which projects upwardly from within a cylindrical cavity in a plastic housing 8 that is open at the top. The canister 7 includes a nozzle which is received in and permanently held by an atomizing socket 10. When the content of the canister 7 is depleted, the canister and the housing, along with the electronic counting circuitry to be described, are discarded.

FIG. 2 is an exploded view of the medication dispensing system and shows the key components of the system including: a plastic enclosure 1 that housed the electronic components including an LCD screen 2, a populated printed circuit board (PCB) 3, a custom microprocessor 4, an activation switch 5, an audio buzzer 6, and a battery 13. The enclosure 1 attaches to and forms part of the main housing 8 which defines a hollow cylindrical mouthpiece 11 and the cylindrical cavity that receives the canister 7.

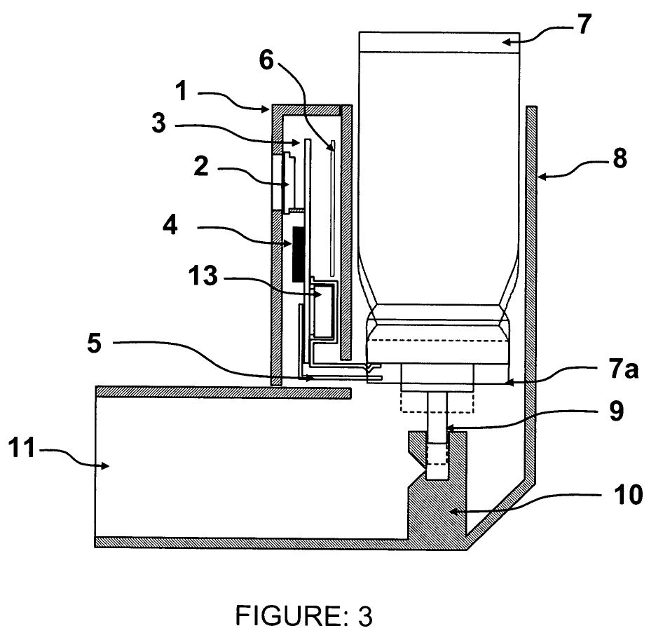

FIG. 3 shows a cross-section of the entire medication monitoring and dispensing system, indicating the positions occupied by electronic enclosure 1, the LCD screen 2, the populated PCB 3, the custom microprocessor 4, the activation switch 5, the audio buzzer 6, the medication canister 7, the battery 13, and the mouthpiece 11. In FIG. 3, the canister 7 is shown in its normal position, and the dashed lines show the medication canister 7 when it is manually depressed to its downward “activation” position. The medication canister 7 is shown with its atomizer 9, loaded into the custom socket 10 that redirects the spray from the medication canister 7, ninety degrees toward the mouthpiece 11.

The activation switch 5 is triggered in one or both of two ways: (1) by closing the distance between the two contact points at the distal ends of the cantilevered conductive arms of the switch 5 when the canister 7 is pressed in the downward direction, and (2) by the metal canister touching the pair of contacts of switch 5 to complete a circuit between the contacts. The electronic circuit on the PCB 3 responds to the closure of the circuit defined by the switch 5 The microprocessor 4 interprets this electronic pulse from either the first or second method of triggering the electronic circuit 3, and sends a digital signal to each and every appropriate segment of the LCD screen 2 to provide an output display which includes an indication to the patient of the correct count of the medication doses remaining in the medication canister 7.

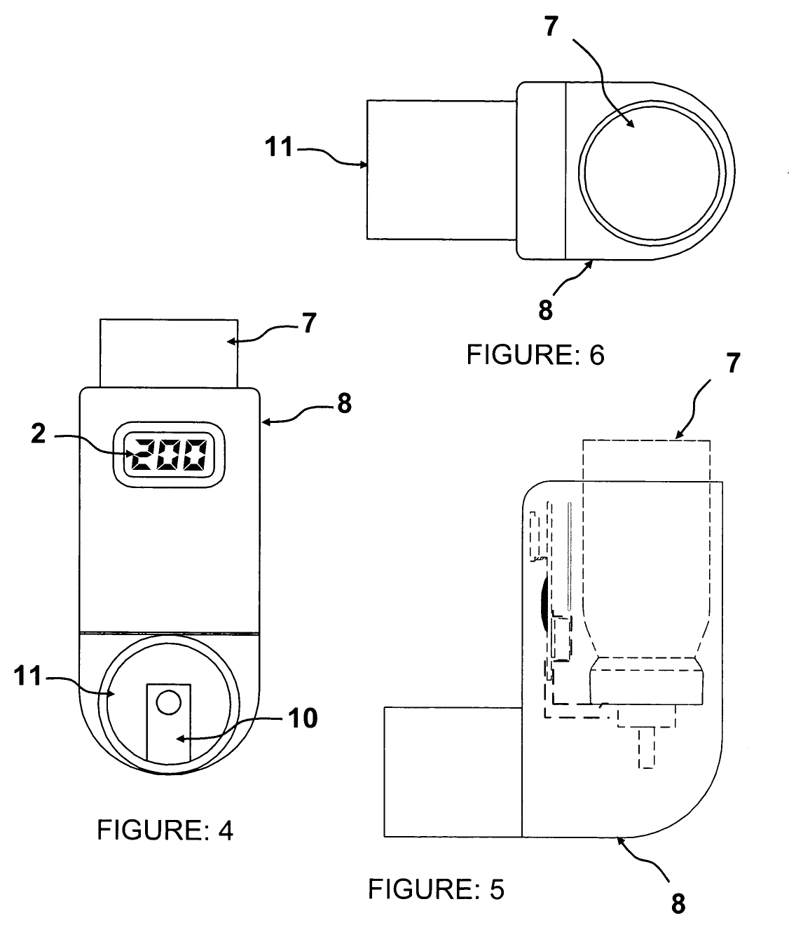

FIG. 4 is a front view profile of the MDI including the trigonometrically shaped housing 8, and the LCD screen 2 which is visible through the electronic enclosure. The medication canister 7, which the user presses downwardly into the housing 8 to dispenses medication from the atomizer in the canister socket 10 into and through the mouthpiece 11.

FIG. 5 is a side view profile of the MDI's plastic housing 8 showing the position of the medication canister 7. FIG. 6 is the top view of the MDI ergonomically designed plastic housing 8 showing the medication canister 7 inserted into the housing 8 and the location of the mouthpiece 11.

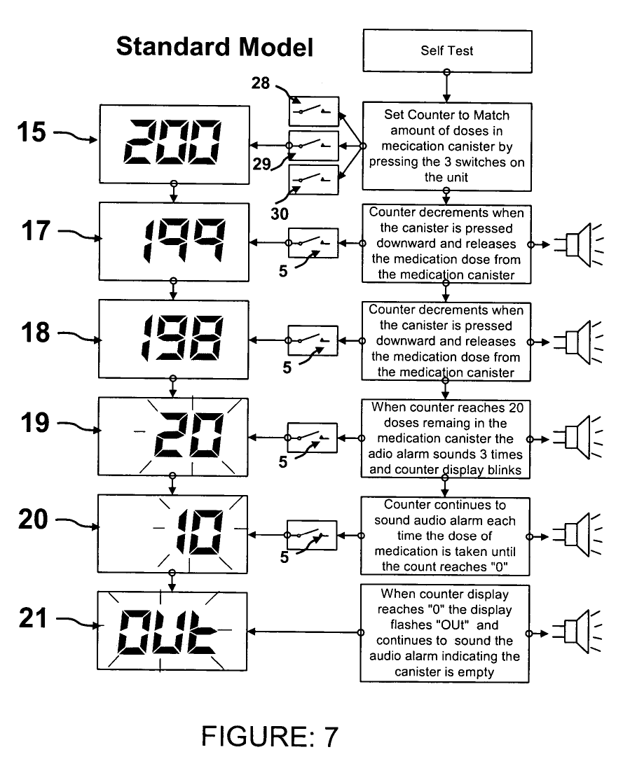

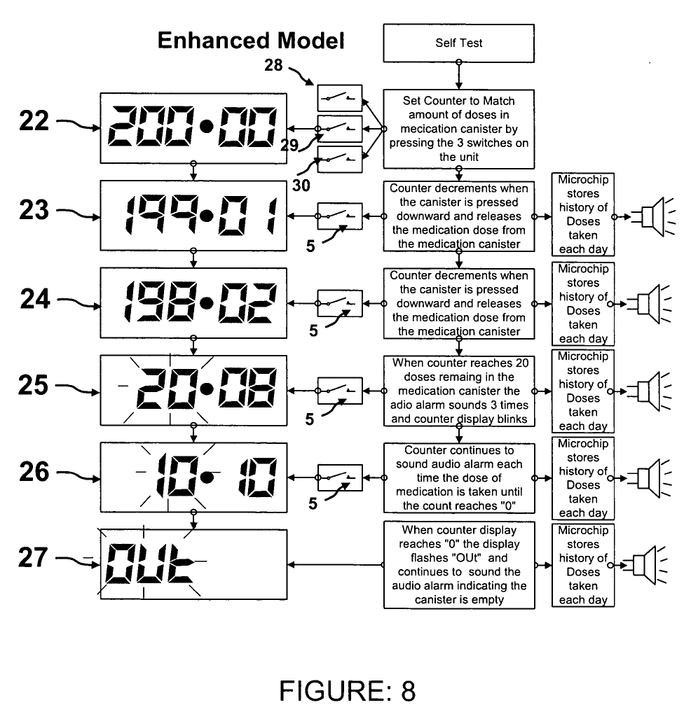

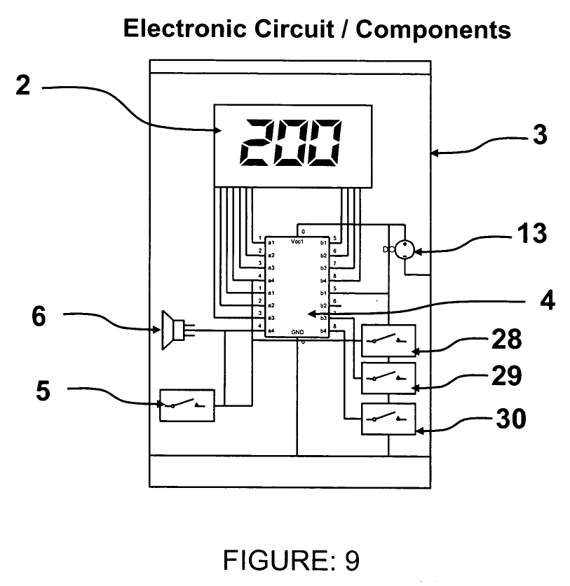

FIGS. 7 and 8 illustrate the sequence of steps performed during regimen for setting, recording and displaying the medication canister dose count throughout the entire monitoring process. The dose count on the LCD screen 2 is set to match the number of doses in the medication canister when the disposable dispenser is assembled. A special sequence of pressing the count presetting switches 28, 29, 30 sets the count held by the electronic counting circuit. The count presetting switches are mounted on the printed circuit board PCB 3 as shown in FIG. 9. The electronic circuitry may be the same as the circuitry described in the above-noted Samiotes et al. U.S. Pat. No. 5,505,192, permitting the PCB 3 and its components to be used in the dispenser shown in that patent or in the “disposable” dispenser contemplated by the present invention. The user-operated count presetting switches and the history switch are not accessible to the user in the current design but are instead placed on the PCB and set at the time the canister and housing are assembled.

The electronic counting circuitry on the PCB 3 activates the LCD 2 to display the correct number of doses 15 in the medication canister 7. When the activation switch 5 is closed by the motion of the canister 7 in a downward motion, the switch 5 sends an electronic pulse to the custom microprocessor 4, and the custom microprocessor 4 then sends a digital code to the segments of the numbers on the LCD screen 2, the number on the LDC screen 2 decrements down each time the activation switch 5 is engaged as shown in 17 and audio alarm 6 sounds once. This process will continue each time a dose of medication and the canister 7 triggers the activation switch 5 until the count on the LCD screen 2 display reaches “20” corresponding to the exact number of doses remaining in the medication canister 7. At this count, the LCD screen 2 flashes and the audio alarm 6 sounds 3 times. This process of display number decrementing and alarm sounding continues until the display 2 reaches “10” as shown in 20, at this point the display will flash and the audio alarm 6 will sound 6 times. This process of display number decrementing and audio alarm 6 sounding continues until the LCD screen 2 reaches “0”; at this point the LDC screen 2 will flash “out” as shown in 21 and the audio alarm 6 will sound 30 times.

The dose count on the LCD screen 2 at the factory or point of assembly when the canister housing 8 and the canister 7 are assembled. The count is set to match the number of doses in the medication canister 7. The manufacturer of the medication canister prints the amount of doses of medication on the canister 7 label. A special sequence of pressing the set switches 28, 29, 30 at the time of assembly activates the LCD screen 2 to display the correct number of doses 15 in the medication canister 7. The presetting switches on the PCB board permit the electric counting circuitry to be assembled with canisters of different capacity; thereby forming factory preset disposable dispensers of different capacities. When the activation switch 5 is closed by the motion of the canister 7 in a downward motion, the switch 5 sends an electronic pulse to the custom microprocessor 4, and the custom microprocessor 4 then sends a digital code to the segments of the numbers on the LCD screen 2, the number on the LCD screen 2 decrements down each time the activation switch 5 is engaged as shown in 23 and audio alarm 6 sounds once. A history of daily doses taken is displayed on the LCD screen as shown in 23. The history of doses are stored in the microprocessor 4 memory for 60 days and each day's history can be retrieved and displayed on the LCD screen. This process will continue each time a dose of medication and the canister 7 triggers the activation switch 5 until the count on the LCD screen 2 reaches “20” corresponding to the exact number of doses remaining in the medication canister 7. At this count, the LCD screen 2 flashes and the audio alarm 6 sounds 3 times. This process of display number decrementing and audio alarm 6 sounding continues until the LCD screen 2 reaches “10”, at this point the LCD screen 2 will flash and the audio alarm 6 will sound 6 times. This process of display number decrementing and audio alarm 6 sounding continues until the LCD screen 2 reaches “0”; at this point the LCD screen 2 will flash “out” as shown in 27 and the audio alarm 6 will sound 30 times. The warning will alert the patient not to use the empty canister any further. This is crucial since the medication canister 7 may feel still feel like it has something in it but this is only propellant and NOT medication.

As seen in the FIG. 8, the LCD preferably displays both the number of doses remaining in the canister 7 and the number of doses that have been previously administered on a given day. Thus, when the disposable dispenser is first purchased and placed in use, it shows the display seen at 22 in FIG. 8, indicating that 200 doses remain in the canister and zero doses have been administered on this day. After the first dose is administered, the counts shown at 23 are displayed, indicating that 199 doses remain and one dose has been administered on the current day. Much later, the display seen at 25 indicates that only 20 doses remain in the canister, and eight doses have been previously administered on that day. When only 20 doses remain, the LCD display of the doses remaining flashes for emphasis to inform the user that the canister is nearly empty. Note that, in the arrangement seen in FIG. 8, it is not necessary to employ a “history” button to display the number of doses already administered on a given day, because that number is always displayed along with the doses remaining count.

### CONCLUSION

It is to be understood that the preferred embodiment which has been described is merely one illustrative application of the principles of the invention. Numerous modifications may be made by those skilled in the art without departing from the true spirit and scope of the invention.

What is claimed is:

1. A disposable medication dispenser comprising, in combination,
a canister for storing medication,
an electronic circuit for displaying the number of doses remaining in said canister, and
a housing for said electronic circuit permanently attached to said canister whereby said housing and said electronic circuit are discarded with said canister when the medication in said canister have been depleted.

2. A disposable medication dispenser as set forth in claim 1 wherein said disposable medication dispenser is an inhaler and wherein said housing permanently attached to said canister defines a mouthpiece through which said medication is dispensed, and wherein said electronic circuit includes a numerical display positioned on the exterior of said housing above said mouthpiece for direct viewing by a user of said dispenser when said mouthpiece is brought to the user's mouth.

3. A disposable medication dispenser as set forth in claim 2 wherein said numerical display shows the number of doses administered from said canister during an immediately preceding time interval.

4. A disposable medication dispenser as set forth in claim 3 wherein said immediately preceding time interval is one day.

5. A disposable medication dispenser as set forth in claim 1 wherein said electronic circuit includes one or more count presetting switches for presetting a count in said electronic circuit at the time said housing and said canister are combined during initial assembly.

6. A disposable medication dispenser as set forth in claim 5 wherein said one or more switches are positioned within said housing and are inaccessible to a user of said dispenser after said dispenser has been assembled.

7. A disposable medication dispenser as set forth in claim 6 wherein said disposable medication dispenser is an inhaler and wherein said housing permanently attached to said canister defines a mouthpiece through which said medication is dispensed, and wherein said electronic circuit includes a numerical display positioned on the exterior of said housing above said mouthpiece for direct viewing by a user of said dispenser when said mouthpiece is brought to the user's mouth.

8. A disposable medication dispenser as set forth in claim 7 wherein said numerical display shows the number of doses administered from said canister during an immediately preceding time interval.

9. A disposable medication dispenser as set forth in claim 8 wherein said immediately preceding time interval is one day.

10. A disposable medication dispenser as set forth in claim 5 wherein said electronic circuit includes a counting switch positioned to sense the movement of said canister when medication is being dispensed from said canister, said counting switch being connected to decrement the displayed number of doses remaining in said canister by one each time a dose of medication is dispensed from said canister.

11. A disposable medication dispenser as set forth in claim 10 wherein said count presetting switches and said counting switch are positioned with said housing and are inaccessible to the user of said dispenser.

12. A disposable medication dispenser as set forth in claim 11 wherein said disposable medication dispenser is an inhaler and wherein said housing permanently attached to said canister defines a mouthpiece through which said medication is dispensed, and wherein said electronic circuit includes a numerical display positioned on the exterior of said housing above said mouthpiece for direct viewing by a user of said dispenser when said mouthpiece is brought to the user's mouth.

13. The method of dispensing medication comprising, in combination, the steps of:
presetting an electronic counting circuit at a count equal to the number of doses of medicine contained in a medication container;
permanently attaching said counting circuit to said container to form a disposable unitary product consisting of both the container and the electronic counting circuit,
using said disposable product to dispense medication until said counting circuit indicates that no doses of medication remain in said container, and thereafter
disposing of said disposable unitary product.

14. The method of dispensing medication as set forth in claim 13 wherein said counting circuit includes a visible display for indicating to a user of said disposable product the number of doses of medication remaining in said container.

15. The method of dispensing medication as set forth in claim 13 wherein said counting circuit includes an audible alarm for indicating to the user when only a limited number of doses of medication remain in said container.

16. The method of dispensing medication as set forth in claim 13 further comprising the step of presenting to the user a visual display of the number of doses remaining in said container each time said medication is dispensed.
